(19) United States
(12) Patent Application Publication
(10) Pub. No.: US 2010/0144003 A1
(43) Pub. Date: Jun. 10, 2010

(54) INDUCTION OF MICROBIAL SECONDARY METABOLITES

(75) Inventors: Andrew Meanrs Spragg, Argyll (GB); Liming Yan, Argyll (GB); Karen Jukes, Argyll (GB)

Correspondence Address: DAVIS WRIGHT TREMAINE LLP/Los Angeles, 865 FIGUEROA STREET, SUITE 2400, LOS ANGELES, CA, 90017-2566, US

(73) Assignee: AQUAPHARM BIO-DISCOVERY LIMITED, Argyll (GB)

(21) Appl. No.: 12/523,700

(22) PCT Filed: Jan. 18, 2008
(86) PCT No.: PCT/GB2008/000149

§ 371 Date: Feb. 2, 2010

(30) Foreign Application Priority Data
Jan. 19, 2007 (GB) 0701021.8

(51) Int. Cl. C12N 13/00; C12N 1/00; C12N 1/20; C12N 1/14; C12N 1/10

(52) U.S. Cl. 435/173.1; 435/243; 435/252.1; 435/254.1; 435/258.1; 435/253.5; 435/253.3

(57) ABSTRACT

The present invention relates to the production of secondary metabolites from microorganisms. In particular, there are provided methods for inducing the rapid production of such compounds from a variety of microorganisms.

### FIELD OF THE INVENTION

The present invention relates to the production of secondary metabolites from microorganisms. In particular, there are provided methods for inducing the rapid production of such compounds from a variety of microorganisms.

### BACKGROUND

When isolated from their natural environment and cultured in planktonic suspension shake flasks, microorganisms can sometimes switch off the ability to produce secondary metabolites. Such agitated suspension cultures in closed flasks provide artificial growth conditions which are not representative of those encountered naturally.

It has previously been shown (Yan et al., 2003), that in the case of two Bacillus sp. (B. pumilus & B. licheniformis), the formation of a biofilm and direct exposure to the air, facilitated the production of antibiotic compounds.

In the natural environment, microorganisms such as, for example, bacteria and fungi, may grow as biofilms attached to surfaces (Lappin-Scott et al., 1995). The growth conditions within a biofilm are usually heterogeneous, for example, pH gradients may develop around the micro-colonies comprising the biofilm (Wimpenny et al., 2000), and limitations in the transport of nutrients and substrates into the biofilm can result in differential starvation of the microorganism(s) (James et al., 1995, Batchelor et al., 1997, Li et al., 2001). Thus, the metabolic processes which occur within microorganisms growing as a biofilms can be markedly different from the metabolic processes which occur in the same organisms when grown as, for example, a planktonic suspension culture.

Secondary metabolites such as those detailed above, represent an important class of compounds with a wide variety of important applications. Notably a number of secondary metabolites have anti-infective properties and antibiotics play a huge role in the treatment of a number of infections. The emergence of multi-drug resistant microorganisms has necessitated further research into the identification of new classes of antibiotic as well as the development of variants of known compounds with increased activity. Thus, the screening of secondary metabolites produced by microorganisms is an indispensable way of obtaining valuable bioactive compounds.

The regulation of secondary metabolite production is complicated and the biosynthetic pathways of most secondary metabolites are not fully understood. It is known that stress-induced networks and numerous cellular systems control the production of secondary metabolites by microorganisms. For example, the limiting or exhausting of nutrients, the biosynthesis of an inducer and/or the decrease in the rate of growth of a microorganism, are all thought to influence the production of secondary metabolites. Such factors are thought to cause a series of signals to be generated which affect a cascade of regulatory events resulting in chemical differentiation (secondary metabolism) and morphological differentiation (morphogenesis).

Some primary metabolites are known to increase the production of secondary metabolites. For example, leucine affects bacitracin synthesis in Bacillus sp., and methionine promotes aminoadipyl-L-cysteinyl-D-valine (ACV) synthetase

To date there has been very little research into the effect of growth in biofilms on the production of secondary metabolites, and there is an increasing need for the development of new anti-infective agents which are effective against the large number of pathogens which exhibit resistance to today's antibiotics.

The present invention is based upon the observation that microorganisms established as a compulsory sessile community (biofilm), under a first set of conditions, may produce secondary metabolites, including compounds with anti-infective activity, when subsequently exposed to an altered set of conditions.

### SUMMARY OF THE INVENTION

In a first aspect of the present invention, there is provided a method of inducing a microorganism to produce secondary metabolites, said method comprising the steps of:
(a) establishing a biofilm, comprising said microorganism, by growth under a first set of conditions; and
(b) altering said first set of conditions such that one or more microorganisms within the biofilm are induced to produce a secondary metabolite(s).

It is to be understood that the microorganism will not generally produce said secondary metabolites unless exposed to said altered set of conditions. The biofilm when grown under said first set of conditions may produce a different secondary metabolite(s) but limited or substantially none of the secondary metabolite(s) produced under said altered set of conditions.

Secondary metabolites comprise many classes of compound and may include, for example, pigments, anti-infective compounds such as antibiotics, antibacterials, antivirals, antifungals and antiprotozoans. In addition, secondary metabolites may also include toxins, effectors of ecological competition and symbiosis, pheromones, enzyme inhibitors, immunomodulating agents, receptor antagonists, pesticides, anti-tumour agents and growth promoters of animals and plants. In the present case, it is particularly desirable to use the methods of the invention to induce the production of anti-infective compounds such as those detailed above. Thus, in a preferred embodiment, the present invention provides a method of inducing a microorganism to produce anti-infective compounds.

Biofilms are well known in the art and may be taken to comprise a community of microorganisms which have colonised a surface or substrate. Microorganisms such as bacteria, fungi, protozoa and yeast, are all capable of forming biofilms. A biofilm may comprise a single species of microorganism or, in some cases, may comprise more than one species of microorganism. The microorganisms within a biofilm are sessile, i.e. attached to a surface or substrate, and as such, biofilms may also be referred to as “compulsory sessile communities”.

Accordingly, the methods described herein may be used with a variety of microorganisms and these may include species of bacteria, fungi or protozoa. By way of example, microorganisms such as Pseudolateromonas, Streptomycetes (Actinomycetes), Berundimonas, Dietzia, Rhodococcus, Micrococcus, Pseudomanas, Serratia, Flavobacteria, Vibrio and Alteromonas sp. may, in accordance with the methods described herein, be induced to produce secondary metabolites.

The growth of microorganisms generally comprises four stages known as the lag, growth (exponential), stationary and death phases. The lag phase is the initial stage in which the microorganisms are preparing to begin growth. During this phase, a number of the microorganisms will not survive and the total number of viable microorganisms may fall, hence the term “lag”. After a short period, those microorganisms which have persisted or survived, multiply and this begins the growth (exponential) phase. This phase sees a rapid increase in the number of microorganisms and, as the available nutrients and space for growth begin to deplete, growth enters the stationary phase. Generally, during the stationary phase, growth begins to slow and there is little' or no further increase in the numbers of microorganisms present. The microorganisms may remain in this phase however, changes in the conditions, i.e. exhaustion of nutrients, accumulation of toxic substances or the like, may cause the microorganisms to begin to die and thus growth enters the death phase.

Microorganisms within a biofilm may be regarded as being in stationary phase, having previously progressed through the lag and growth phases. Advantageously therefore, the first conditions of the present invention may be taken to be conditions which permit initial growth and progression of said microorganisms to the stationary phase and the establishment of a biofilm.

Accordingly the term “first conditions” may refer to growth parameters, for example the choice of growth medium, or the temperature and/or pressure at which a microorganism is grown, cultured or maintained. Generally, in order to establish a community or biofilm of microorganisms, it is desirable use a liquid, semi-solid or solid medium, rich in nutrients, and which is suited to the growth of a particular microorganism. In one embodiment of the present invention the microorganism may be grown or cultured on a surface or substrate, a portion of which is brought into contact with a growth medium.

Those familiar with microorganism culture techniques will be aware of the types of media that may be used, and it should be noted that, for brevity, only a limited number are mentioned herein. For example, microorganisms isolated from the marine environment may be cultured on a designated “marine” medium, while those organisms isolated from, for example, the mammalian gut, may be cultured on a medium which selectively permits the growth of such organisms, such as MacConkey agar or broth. Such media may comprise, for example NaCl, bile salts and other compounds and components intended to replicate the conditions found in the natural environment of the organism in question.

Other media may have a more general utility, and may be used to culture a number of different organisms isolated from different environments. Such media may be known as “general purpose media” and may include, for example, blood, chocolate, columbia, LB, nutrient and potato-dextrose (PD) agars and broths. These media may be further supplemented with any chosen agent, compound or substance to facilitate the growth of a particular microorganism, or to impart a degree of specificity to the medium. For example, potato dextrose medium may be further supplemented with, for example, a yeast extract (PDY media). For example the media may be supplemented with between about 0.1 and 1% (w/v) yeast extract, more preferably about 0.2% (w/v).

In addition, the term “first conditions” may include compounds, for example proteins or peptides, amino acids, nutrients for example vitamins, nucleic acids or other small organic molecules. Such compounds may, for example, be added to the chosen growth medium or additionally, or alternatively, directly to the microorganisms.

Advantageously the “first conditions” facilitate the establishment of a biofilm within about 1 to about 10 days. Preferably, a biofilm should establish within about 2 days to about 5 days, and more preferably within about 3 to about 4 days.

Preferably, the biofilm is established on a particular surface or substrate. Advantageously, the surface or substrate (referred to hereinafter as the “substrate”), upon which the biofilm is to be established, is unable to be metabolised by the microorganism(s) of the biofilm, and may thus be referred to as an “inert” material or substance. Therefore, in a preferred embodiment of the present invention, a biofilm comprising a particular microorganism or microorganisms, is established on an “inert substrate”.

Preferably, in addition to being inert, the substrate is a semi-permeable material or substance. Suitable inert, semi permeable substrates upon which a biofilm may be established include, but are not limited to, glass fibre, nylon and cellophane membranes. Alternatively, the biofilm may be established on a substrate which comprises a regenerated cellulose or cellulose ester material, for example material suitable for dialysis procedures such as Visking dialysis tubing.

It should be noted that the choice of substrate upon which a biofilm is to be established, may depend upon the microorganism(s) of the biofilm as some substrates, although unable to be metabolised by certain microorganisms, may be metabolised by others. For example, bacteria such as the Actinomycetes may cause the degradation of substrates which comprise nylon. Accordingly, biofilms which comprise an Actinomycete, should be cultured on a substrate which does not comprise nylon. In addition, while it is desirable that the substrate be semi-permeable, it should not be so permeable so as to allow the passage of the microorganism(s) of the biofilm through the substrate. For example, certain bacteria may be able to pass through the pores present in substrates which comprise, for example, materials such as glass-fibre. The skilled addressee can easily chose a semi-permeable substrate or suitable pore size dependent on the microorganism(s) used to form the biofilm.

In accordance with the present invention, the inert, semi-permeable substrate is maintained under a first set of conditions in order to establish a biofilm. For example the substrate may be placed on to the surface of a sterile growth medium. Advantageously, the substrate may be retained in position on the surface of the sterile medium by surface tension. Additionally, or alternatively, the material or substance may be retained in position by some other means, for example by some form of support structure. Alternatively, the substrate may be retained in position by a combination of surface tension and some other means, for example a support structure. In this way, one surface of the substrate is in contact with the sterile growth medium while the opposing surface is in contact with the air.

The substrate upon which the biofilm is to be established, may be inoculated with a chosen microorganism(s) by any suitable means for example, by means of a swab, either before or after exposure to said first set of conditions.

The substrate may be formed into any particular shape, for example, the substrate may take the form of a disc or other essentially 2-dimensional shape.

Alternatively the substrate may take a 3-dimensional form and may, for example, comprise a plurality of hollow tubes, folds or cavities which may serve to increase the surface area over which a biofilm may be established.

An exemplary biofilm culture system is detailed in the paper by Yan et al., 2003. The system described therein provides an air-membrane surface (AMS) reactor which permits the establishment of a compulsory sessile microbial community (biofilm) on a selected substrate. Briefly, the chosen substrate is first placed on the surface of a volume of sterile liquid semi-solid growth medium where it is held in place by surface tension. As such only one surface of the substrate is in contact with the sterile growth medium while the opposing surface is exposed to the air. The surface of the substrate which is exposed to the air is then inoculated with the microorganism(s) which are to form the biofilm. The limited availability of “free” growth medium facilitates the establishment of a biofilm or compulsory sessile community.

The conditions under which a particular microorganism produces secondary metabolites may differ from those required to establish a biofilm comprising said microorganism. Thus, in accordance with the methods of the present invention, in order to induce the production of secondary metabolites, the microorganisms comprising the biofilm are subjected to a second or altered set of conditions. For example, the altered conditions required to induce the production of secondary metabolites may include or comprise toxic/damaging agents or conditions or compounds which may inhibit, restrict or prevent the growth or survival of a microorganism. Generally, the altered conditions which induce the production of secondary metabolites may be said to place the microorganisms of the biofilm under stress. Crucially however, said altered conditions are tolerated by microorganisms which have already been established as a biofilm under, for example, the abovementioned first conditions.

Thus the “first conditions” support the microorganism(s) during the lag and growth phases and permit the maintenance of an established biofilm, while the “second” or “altered conditions” may be unsuitable to support the growth of the microorganism, but are tolerated by at least a portion of the microorganisms established as a biofilm. In this way, microorganisms which would otherwise fail to readily establish a biofilm under the conditions required to induce the production of secondary metabolites, may be induced to do so by the method of the present invention which provides two sets of conditions, a first set facilitating the rapid establishment of a biofilm and a second or altered set, to induce the production of secondary metabolites.

The methods described herein provide a two-step process for inducing a microorganism to produce secondary metabolites, wherein the first step comprises establishing a biofilm as substantially described above, and the second step comprises altering the conditions to induce the microorganism(s) to produce secondary metabolites.

The “altered conditions” which induce secondary metabolite production may include the use of particular growth conditions or compounds which modulate the primary and/or secondary metabolism of a microorganism. For example, such compounds may include those capable of modulating microbial stress-induced network pathways. Thus, for example, once a biofilm has been established, the biofilm may be maintained under conditions or in the presence of compounds which induce secondary metabolite production. The altered conditions of the present invention may include the use of

compounds such as primary metabolites or nutrients which induce the production of secondary metabolites. It should be understood that the terms “primary metabolites” or “nutrients” may be taken to include, for example, vitamins, for example vitamin K or its synthetic equivalent, menadione (vitamin K3), carbohydrates, proteins or peptides, amino acids and other similar compounds. In addition, “primary metabolites” or “nutrients” may also refer to, for example, nucleic acids, minerals and metal ions, for example ferric, manganese and/or cupric ions. Metal ions may be added in the form of, for example, any organic or inorganic metal salt, for example ferric citrate or ferric chloride. Advantageously the metal ions may be added to a final concentration of about 1 to about 10 mM, preferably 1-5 mM and more preferably 1-2 mM.

Additionally, or alternatively, the altered conditions may include altered growth media. Nutrient limitation and/or exhaustion may have an effect upon the primary and/or secondary metabolism of microorganisms and as such may induce the production of secondary metabolites. Accordingly, a particular growth media may be adapted to limit the availability of certain nutrients to the microorganism. This may be achieved by providing a medium which lacks a certain component or components which are essential to the biological systems of the microorganism. For example, the medium may lack certain nutrients, such that microorganisms maintained thereon are starved or deprived of said nutrient.

The altered conditions which induce the production of secondary metabolites may also comprise or include the addition of agents or compounds such as, for example, antibiotics, antifungals, antivirals or the like, which may induce stress responses in microorganisms. Examples of antibiotics which may function in this manner include the quinolones, for example ciprofloxacin. Furthermore, compounds capable of altering osmotic conditions and oxidative compounds or molecules are further recognised as capable of inducing secondary metabolite production. Examples of oxidative compounds include, for example hydrogen peroxide or reactive oxygen generators such as menadione or paraquat. In the case of menadione, the quinine structure gives one electron to an oxygen molecule and is oxidized to semiquinone, and semiquinone can further give another electron to another dioxygen and is oxidized to hydroquinone. Therefore, during the process of quinone oxidization to hydroquinone, two molecules of superoxide will form. The superoxide places the microorganisms of the biofilm under stress and induces the production of secondary metabolites.

Additionally or alternatively, compounds capable of inducing the production of secondary metabolites may be added directly to a microorganism or as a component of a substrate upon which they are cultured.

The altered conditions may also include certain environmental conditions or factors which have the effect of inducing the production of secondary metabolites.

For example, the altered conditions may include subjecting the microorganism(s) to radiation, for example ionising radiation and/or electro-magnetic radiation, temperature and/or pressure variations. In the case of exposure to electro-radiation, the microorganism(s) may be subjected to short wavelength electromagnetic radiation or ultraviolet radiation, of between about 100 to about 400 nm, preferably 200-300 nm and more preferably 254 nm. With regards ionising radiation, the microorganism(s) may be subjected to, for example, alpha, beta, gamma and/or x-ray radiation.

Thus, in a second aspect of the present invention, there is provided a method of inducing a microorganism to produce secondary metabolites, said method comprising the steps of:
(a) establishing a biofilm, comprising said microorganism, by growth under a first set of conditions; and
(b) altering said first set of conditions such that one or more microorganisms within the biofilm are induced to produce a secondary metabolite;
   wherein the altered conditions comprise exposing the microorganism to radiation.

The length of time for which a microorganism or microorganisms may be exposed to radiation may vary depending on the microorganism(s) used. By way of example however, it may be desirable to repeatedly expose the microorganism(s) to radiation over a period of about 1 to about six days, preferably 2 to five days and more preferably four days. Furthermore, the duration of each exposure to radiation may vary, and by way of example, microorganisms may be exposed to about four to about 20 hours of radiation, preferably 6 to about fifteen hours and more preferably 12 hours.

It should be noted that any one of the abovementioned conditions, compounds or agents may be used either alone or in combination with any other condition, compound or agent to create the altered conditions which induce the production of secondary metabolites. For example, in one embodiment of the present invention, the altered conditions for induction of secondary metabolite production may include the use of menadione in combination with ferric, manganese and/or cupric ions. Alternatively, and in a further embodiment of the present invention, the altered may include the use of nutrients such as menadione and compounds such as hydrogen peroxide, together with either ferric, manganese or cupric ions.

### DETAILED DESCRIPTION

The present invention will now be described in detail and with reference to the following figures which show

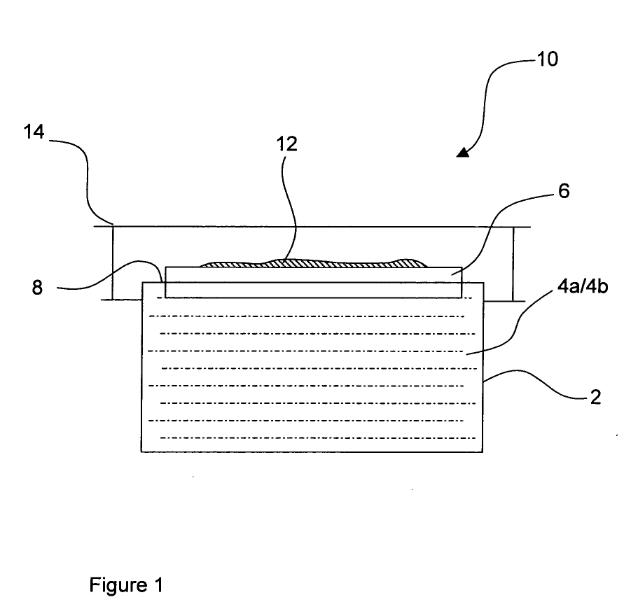

FIG. 1: An air-membrane surface bioreactor system for use in a method according to the present invention, generally designated by reference numeral 10 as described by Yan et al., 2003. The bioreactor 10 comprises a chamber 2 which holds a volume of growth medium 4 and which supports growth substrate 6 via surface tension. The substrate 6 comprises an inert, semi permeable material which is partly submerged in the growth medium 4 and partly exposed to the air and as such provides a air/surface interface shown by reference numeral 8. In the embodiment shown, a biofilm 12 has been established on the surface of the substrate 6 which is exposed to air. The chamber 2 is sealed by means of lid 14 which prevents contamination of the growth substrate 6. Once the biofilm 12 has been established, the growth medium 4 is replaced with an altered medium 4b, which induces the production of secondary metabolites. The secondary metabolites pass through the inert, semi permeable substrate and accumulate in the altered medium 4a.

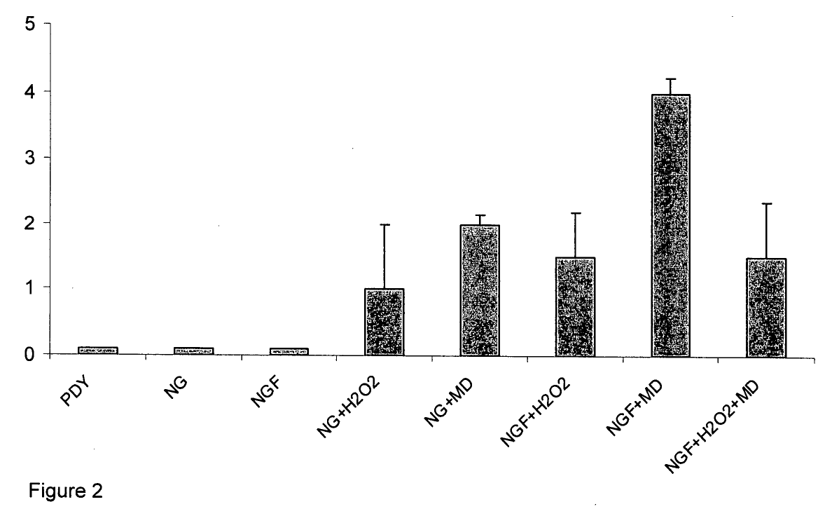

FIG. 2: The effect of oxidative stress on the elicitation of antimicrobial compounds produced by Streptomyces sp. AQP274. The figure suggests that hydrogen peroxide was able to elicit the production of antimicrobial compounds in some culture systems. In contrast, the induction effect of menadione was more stable and more preferable according to this invention, due to the less standard deviation. Briefly, both menadione and hydrogen peroxide were able to elicit the production of antimicrobial compounds in the genus actinomycetes, more preferably in Streptomyces sp. Medium formulation was as follows: “PDY”, potato dextrose with yeast extract; “NG”, nutrient broth with 1% (v/v) glycerol; “NGF”, nutrient broth containing 1% (v/v) glycerol and 1 mM ferric citrate; “H₂O₂”, hydrogen peroxide; “MD”, menadione.

Microbial cultivation in the different media was carried out in quadruplicate, and standard deviation was indicated by the error bar.

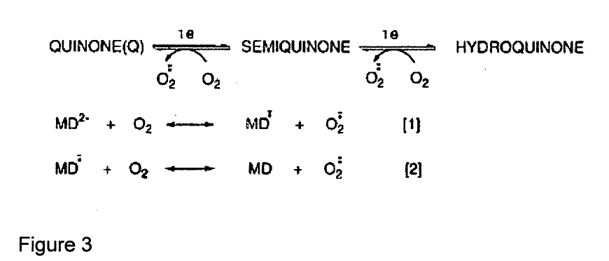

FIG. 3: Proposed superoxide generation by autoredox reaction of quinone group in menadione. The quinone structure in menadione gives one electron to oxygen and is oxidized to semiquinone, and semiquinone can further gives another electron to another dioxygen and is oxidized to hydroquinone. Therefore, during the process of quinone oxidization to hydroquinone, two molecules of superoxide will form.

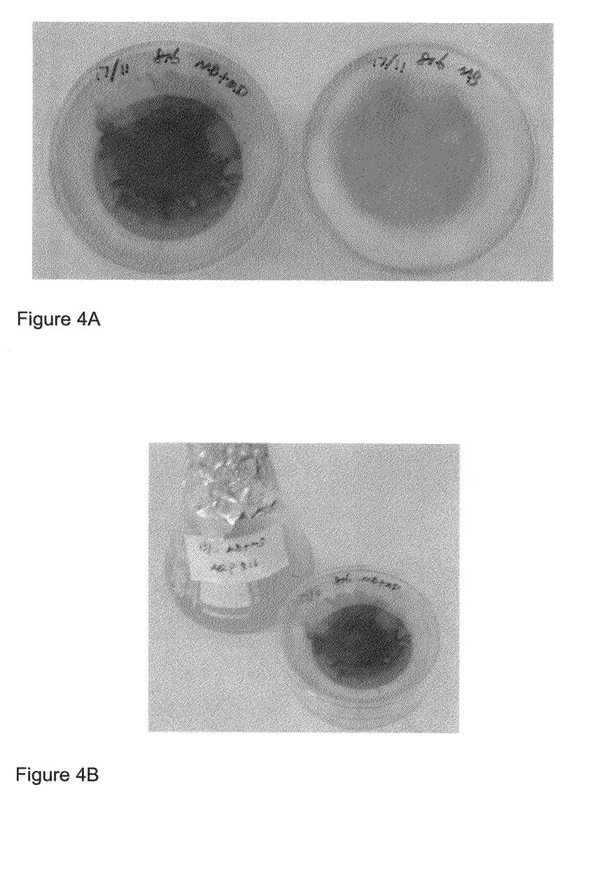

FIG. 4A Elicitation of pigment production by a marine Pseudoalteromonas sp. strain AQP816. The effect of menadione on the dark pigment production when AQP816 was grown using nylon membrane culturing system. The significant production of a dark pigment was observed when menadione was added in the media. FIG. 4B. The effect of nylon membrane surface culturing system on dark pigment production by AQP816 in the same media (marine broth containing 100 μg/ml menadione). The pigment was only likely to produce when AQP816 was grown using membrane surface culturing system.

### EXAMPLES

### Example 1

Two-Step Cultivation Approach to Grow Micro-Organisms

Media for the growth of certain bacteria is not necessarily ideal for the production of secondary metabolites. Therefore a two-step cultivation approach was applied that elicited production of secondary metabolites in bacteria, more preferably of the genus actinomycetes that were previously not produced under normal shake flask culture conditions. Using either planktonic shake flasks, more preferably, the Glass Fibre Membrane Bio-Film Culturing System, suitable micro-organisms were inoculated into a growth medium until an adequate microbial community was established. At this point, the biomass or biofilm was transferred to another growth medium which was appropriate for the production of secondary metabolites.

More preferably, an Actinomycete Streptomyces sp. strain AQP274 when inoculated onto a glass fibre membrane was able to produce sufficient biomass within 4 days when grown on a medium containing Potato Dextrose agar containing 0.2% (w/v) Yeast extract (PDY), however when screened for the production of antimicrobial compounds, this strain showed no detectable antibiotic activities when screened against MRSA when cultured in this medium. At the same time using the same culture methods, AQP274 grew very slow in media containing Nutrient agar (28 g/L), glycerol (1% v/v), 1 mM ferric citrate and menadione (0.15 g/L), termed medium NGFM, however, after cultivation at room temperature for 21-24 days, the antimicrobial activity against an MRSA strain and against a Candida albicans strain could be easily detected. More preferably, to speed up this process of secondary metabolite production and therefore improve bio-process optimisation using this system to elicit anti-infective compounds from this strain quickly, AQP274 was first inoculated in medium PDY and then transferred to NGFM medium. Results showed antimicrobial activity was produced by this strain against the aforementioned test strains and could be detected at 4 days of growth of the antibiotic producing microbe. Furthermore, the amount of crude medium extract necessary for the detectable activity decreased 2 folds.

### Example 2

### Complex—Establishment of Compulsory Sessile Community (Biofilms) of Micro-Organisms

This two-step cultivation approach was further improved by means of the establishment of compulsory sessile communities of anti-infective producing micro-organisms, more preferably of the genus actinomycetes onto an inert glass fibre membrane. It has been reported that genes associated with antibiotic production in bacilli could be regulated by environmental stresses (Yan et al. 2003). In addition, cells grown within a biofilm or sessile community have developed complicated mechanisms which exhibit more resistance to various types of environmental stresses; therefore they are adapted to more extensive physical and chemical environment in contrast with their planktonic suspension counterparts. However, when grown using planktonic suspension cultivation method, many species do not build up sessile microbial matrices on surface of inert support automatically; therefore, a compulsory sessile microbial matrix was established at air-solid/liquid interface. Pure or mixed microbial strain(s) for example bacteria or fungi, more preferably actinomycetes were inoculated on to the surface of a semi-permeable inert support, such as a nylon membrane or glass fibre filter. The inert support was subsequently placed on top of a medium which allowed the inert support to separate the microbial biomass from the growth media. The biomass was built up at one side of the inert support and the growth media at the other. Due to absence of free liquid, microbial biomass will grow in the form of a compulsory sessile microbial matrix (biofilm) on the surface of this support system. This method can establish a compulsory sessile matrix of any micro-organism more preferably actinomycetes, more preferably on to the surface of a semi-permeable inert support system.

### Example 3

### Elicitation of Antimicrobial Compound Production Using Oxidative Stress

Using the culture system described in example 2, various stresses can be used to elicit production of secondary metabolites by established biomass, more preferably, in compulsory sessile microbial communities.

This invention uses oxidative stress imposed by reactive oxygen species (ROS) that can be carried out using peroxide compounds including hydrogen peroxide, or superoxide generators such as menadione or paraquat, with supplementation of transition metal ions such as ferric, manganese or cupric ions in bacteria cultured as a compulsory sessile microbial community. More preferably, Streptomyces sp. strain AQP274 was cultivated using this system in a two-step approach to induce antimicrobial compound production. An initial compulsory sessile matrix (biofilm) was established on a piece of glass fibre filter in PDY medium. A microbial matrix was then established on this filter, which was then subsequently transferred to another medium which imposed oxidative stress, as described above using reactive oxygen generators (ROS), more preferably H₂O₂, or menadione in the presence of ferric and or cupric ions.

The production of antimicrobial compounds using this described system were analysed between various cultures with different medium formulation (FIG. 2). More preferably, all the media were solidified with 0.3% (w/v) agar powder (No.3), which assisted support of the glass fibre filter. More preferably, filter-sterilised menadione and/or H₂O₂ were added to the media when cooled down to approximately 37° C. More preferably, results suggested that both menadione and hydrogen peroxide could elicit antibiotic and anti-fungal compounds into the medium, and ferric and or cupric ions enhanced the production of these secondary metabolites.

Hydrogen peroxide was able to elicit antibiotic production in bacteria and fungi and more preferably in strain AQP274. More preferably, providing a low concentration of hydrogen peroxide (less than 0.5%) was used together with a frequent (more than 3 times per day) supplementation strategy was better for elicitation of antimicrobial compounds. This was shown to be a better system than providing a high concentration of hydrogen peroxide in a single batch treatment. In addition, menadione was more preferable in the elicitation of secondary metabolites from bacteria, more preferably actinomyctes.

Menadione (vitamin K3, 2-methyl-1,4-naphthoquinone) has been extensively used as a model of redox-cycling quinine to study superoxide stress in both prokaryotic and eukaryotic organisms (Fernandes and Mannarino, 2007; Goldberg and Stern, 1976). Quinone redox cycling implies autoxidation of quinone reduction products. During autoxidation, two single-electron transfer steps are accompanied with formation of semiquinone intermediates and superoxide (FIG. 3).

### Example 4

Elicitation of Antimicrobial Compound Production Using UV Light

UV light can cause various stresses and it is well known that UV causes damage to DNA and has been well studied in micro-organisms. In addition, UV can also cause the production of singlet oxygen species, which is another ROS. The culturing system described in example 2 is used to produce antimicrobial compounds in bacteria, preferably in actinomycetes. A Streptomyces sp. strain AQP1159 is cultivated using the GFMS bioreactor system to establish a sessile community matrix at air/glass fibre membrane interface in nutrient broth containing 1% (v/v) glycerol and 1 mM Fe citrate (NGF). After the matrix was built up, the bioreactor was exposed to UV₂₅₄ for 36 hours, 12 hours each day for 3 days consecutively. Then the NGF media beneath the glass fibre membrane was refreshed and the culture was subsequently incubated for 4 days at room temperature. The liquid media beneath the glass fibre membrane was then removed to carry out antimicrobial assay.

Without exposure in UV₂₅₄, AQP1159 did not produce detectable antimicrobial compounds against Candida albicans and MRSA, however, after treatment by UV₂₅₄ and media refreshing, AQP1159 produced antimicrobials against both Candida albicans and MRSA. Using the same media, freshly inoculated AQP1159 without build-up of enough biomass on glass fibre membrane did not grow any more after exposing to the UV₂₅₄. The refreshing of NGF media was also critical for the production of antimicrobial compounds.

### Example 5

Elicitation of Secondary Metabolite Production by a Range of γ-Proteobacteria Using the Bio-Fermenter Designed to Grow Bacteria as a Compulsory Sessile Microbial Matrix (Biofilm).

A range of eubacteria were tested for the induction of secondary metabolites using the described method for culturing bacteria in a sessile microbial community using a free-radical generating media to induce a stress response. For example, a Pseudoalteromonas sp. strain, AQP816 was inoculated on surface of nylon membranes which was subsequently placed on a shallow dish filled with marine broth. When an adequate biofilm of AQP816 had established on the surface of the nylon membrane at the air/membrane interface, the marine broth underneath the membrane was refreshed with various media including fresh marine broth, marine broth containing 100 μg/ml menadione; marine broth containing 3% v/v H₂O₂, marine broth containing 1% (v/v) glycerol and marine broth containing 1 mM Ferric citrate. Results obtained, showed that the addition of menadione could significantly elicit the production of certain dark pigmented compounds using this surface method, compared to no pigment production in the correspondent shake flask cultures (FIG. 4). This observation was also observed in cultures of actinomycetes, streptomycetes, γ-proteobacteria, including brevundimonads, dietzia, rhodococci, pseudomonads, serratia, flavobacteriacea, vibrio & pseudoalteromonads that when grown on a membrane in the presence of menadione could elicit the production of secondary metabolites.

### Example 6

### Elicitation of Secondary Metabolites by a Range of Microorganisms Using Various Agents to Impose Stress

A number of further bacterial and fungal isolates were grown as biofilms essentially as described in Example 2, and various stresses imposed to seek to elicit secondary metabolite production.

Examples of various strains which exhibited significant secondary metabolite change using various stress imposing methods are summarised in Table 1. All the strains were grown within biofilms, among which fungi were able to form natural biofilm. The detection of any secondary metabolite production, which was different from normally produced secondary metabolites for any given strain, was carried out 7 days after the stressing condition was applied.

0.5 mM NaNO₃ was shown to significantly delay growth of most microorganisms in the isolates tested. In addition, many strains also displayed changed morphologies as well as secondary metabolite production when grown in a medium containing 0.5 mM NaNO₃. A Streptomyces sp. strain AQP4511 produced a red orange compound, which has a naphthoquinone structure, in a PDY medium supplemented with 0.5 mM NaNO₃. The compound showed very strong activity against most of Gram-positive bacterial strains.

Heavy metals such as Cu, Fe, Mn have also been used impose stress on many micro-organisms. Cu has been used in paints to prevent biofouling process in marine environment; Fe and Mn can affect the respiration chain of many cells. In one example, AQP1148 which was identified as Bacillus licheniformi, did not produce bacitracin or a red pigment possibly pulcherrimin, unless it was grown within a compulsory biofilm established in direct contact with the air, and in media containing ferric ion and carbohydrates. Mn²⁺ also elicited the production of bacitracin when the strain was grown in a biofilm. The optimised concentration of Fe³⁺ was 1 mM and Mn²⁺ 0.5 mM. Higher than these concentrations had led to a significant slowing in growth which indicated the stress the metals imposed.

TABLE 1
Elicitation of secondary metabolite production under stressed condition
by some isolates in Aquapharm

| | | Secondary metabolites |
|---|---|---|
| Strain Number | Stress imposed | elicited |
| AQP274 | Oxidative | cyclohexamide |
| Streptomyces griseus | (menadione) + H₂O₂ | |
| AQP1159 | UV₂₅₄ | Anti-Candida, MRSA |
| Streptomyces sp. | | |
| AQP1148 | Oxidative (menadione) | Anti-MRSA |
| Bacillus licheniformis | Heavy metal (Fe) | |
| AQP1569 | Oxidative (menadione) | Anti-Staph. aureus |
| Bacillus sp. | | |
| AQP803 | Oxidative (menadione) | Anti-Staph. aureus |
| γ-proteobacteria | | |
| AQP806 | Oxidative (menadione) | Anti-Staph. aureus |
| γ-proteobacteria | | |
| AQP807 | Oxidative (menadione) | Anti-Staph. aureus |
| γ-proteobacteria | | |
| AQP808 | Oxidative (menadione) | Anti-Staph. aureus |
| Flavobacteriaceae sp. | | |
| AQP809 | Oxidative (menadione) | Anti-Staph. aureus |
| γ-proteobacteria | | |
| AQP820 | Oxidative (menadione) | Anti-Staph. aureus |
| γ-proteobacteria | | |
| AQP858 | Oxidative (menadione) | Anti-Staph. aureus |
| γ-proteobacteria | | |
| AQP859 | Oxidative (menadione) | Anti-Staph. aureus |
| γ-proteobacteria | | |
| AQP211 | Heavy metal (Cu + Fe) | Anti-MRSA, E. coli |
| Fungus | | |
| AQP842 | Heavy metal (Mn), | Anti-Candida albicans |
| Streptomyces anulatus | | |
| AQP4511 | Oxidative | Gunacin, (against |
| Streptomyces sp. | (—NO₃ + H₂O₂), | MRSA) |
| AQP816 | Oxidative (menadione) | Brown pigment |
| γ-proteobacteria | | |
| AQP884 | Oxidative (menadione) | Red pigment |
| Flavobacteriaceae sp. | | |

### REFERENCES

Batchelor, S. E., M. Cooper, et al. (1997). Cell density-regulated recovery of starved biofilm populations of ammonia-oxidizing bacteria. Appl Environ Microbiol 63(6): 2281-6.

Costerton, J. W., Z. Lewandowski, et al. (1995). Microbial biofilms. Arum Rev Microbiol 49: 711-45.

Fernandes, P. N., S. C. Mannarino, et al. (2007). Oxidative stress response in eukaryotes: effect of glutathione, superoxide dismutase and catalase on adaptation to peroxide and menadione stresses in Saccharomyces cerevisiae. Redox Rep 12(5): 236-44.

Goldberg, B. and A. Stern (1976). Production of superoxide anion during the oxidation of hemoglobin by menadione. Biochim Biophys Acta 437(2): 628-32.

James, G. A., D. R. Korber, et al. (1995). Digital image analysis of growth and starvation responses of a surface-colonizing Acinetobacter sp. J Bacteriol 177(4): 907-15.

Li, Y. H., M. N. Hanna, et al. (2001). Cell density modulates acid adaptation in Streptococcus mutans: implications for survival in biofilms. J Bacteriol 183(23): 6875-84.

Wimpenny, J., W. Manz, et al. (2000). Heterogeneity in biofilms. FEMS Microbiol Rev 24(5): 661-71.

Yan, L., K. G. Boyd, et al. (2003). Biofilm-specific cross-species induction of antimicrobial compounds in bacilli. Appl Environ Microbiol 69(7): 3719-27.

1-16. (canceled)

17. A method of inducing a microorganism to produce secondary metabolites, said method comprising the steps of:
(a) establishing a biofilm, comprising said microorganism, by growth under a first set of conditions; and
(b) altering said first set of conditions such that one or more microorganisms within the biofilm are induced to produce a secondary metabolite.

18. The method according to claim 17 wherein the secondary metabolite is selected from pigments, anti-infective compounds such as antibiotics, antibacterials, antivirals, antifungals, antiprotozoans, toxins, effectors of ecological competition and symbiosis, pheromones, enzyme inhibitors, immunomodulating agents, receptor antagonists, pesticides, anti-tumour agents and growth promoters of animals and plants.

19. The method according claim 17 wherein said microorganism comprises at least one species of bacteria, fungi, protozoa or mixture thereof.

20. The method according to claim 19 wherein said at least one microorganism includes Pseudoalteromonas, Streptomycetes (Actinomycetes), Berundimonas, Dietzia, Rhodococcus, Micrococcus, Pseudomanas, Serratia, Flavobacteria, Vibrio and Alteromonas sp.

21. The method according to claim 17 wherein when cultured under said first set of conditions, said microorganism(s) is cultured on a surface or substrate, a portion of which is brought into contact with a growth medium.

22. The method according to claim 21 wherein the surface or substrate is inert.

23. The method according to claim 17 wherein the biofilm is established over a period of 1 to 10 days.

24. The method according to claim 21 wherein the surface or substrate is semi-permeable.

25. The method according to claim 24 wherein the semi-permeable surface or substrate is retained on the surface of a sterile growth medium by surface tension or a support structure.

26. The method according to claim 17 wherein the altered set of conditions designed to induce production of the secondary metabolite comprises toxic or damaging agents or conditions, or compounds which inhibit, restrict, or prevent the growth or survival of a microorganism within the biofilm.

27. The method according to claim 26 wherein the altered conditions comprises the addition or administration of at least one of: a vitamin or synthetic equivalent, a carbohydrate, a protein or peptide, an amino acid, nucleic acid, a mineral, a metal or metal ion, nutrient limitation, an antibiotic, an antifungal, an antiviral, a compound capable of altering osmotic conditions, ionising radiation, electromagnetic radiation, altered temperature or altered pressure.

28. The method according to claim 27 wherein the altered conditions comprise the addition or administration of at least one of a metal or metal ion, a compound capable of altering osmotic conditions, and/or electromagnetic radiation.

29. The method according to claim 28 wherein the electromagnetic radiation is UV light.

30. The method according to claim 28 wherein the metal ion is Mn, Cu and/or Fe.

31. The method according to claim 28 wherein the compound capable of altering osmotic conditions is menadione, H₂O₂ and/or nitrate.

32. The method according to claim 27 wherein the altered conditions are maintained for a period of between about 1 to 6 days.
